(19) United States
(12) Patent Application Publication
(10) Pub. No.: US 2004/0059318 A1
(43) Pub. Date: Mar. 25, 2004

(54) Instrument and method for surgical extraction

(75) Inventors: Jeffrey Zhang, Collierville, TN (US); Lukas Eisermann, Memphis, TN (US)

Correspondence Address: HAYNES AND BOONE, LLP, 901 MAIN STREET, SUITE 3100, DALLAS, TX, 75202, US

(73) Assignees: SDGI Holdings, Inc., Wilmington, DE (US); Medtronic Sofamor Danek, Inc., Memphis, TN (US)

(21) Appl. No.: 10/665,578

(22) Filed: Sep. 19, 2003

Related U.S. Application Data
(60) Provisional application No. 60/412,183, filed on Sep. 20, 2002.

(51) Int. Cl. A61B 17/00

(52) U.S. Cl. 606/1

(57) ABSTRACT

The present invention provides an instrument and method for surgical extraction. In one example, a surgical instrument for extracting a prosthetic device includes a distal portion transitionable from an insertion configuration to an extraction configuration, wherein the insertion configuration is adapted for displacement along a portion of a prosthetic device, and the extraction configuration is adapted for engaging and extracting the prosthetic device, and a proximal portion connected to the distal portion. In another example, an instrument for surgical extraction includes at least one extraction prong wherein the at least one extraction prong comprises a transverse flange, and a mounting portion wherein the at least one extraction prong is secured to the mounting portion.

### CROSS-REFERENCE TO RELATED APPLICATION

This invention claims priority to the U.S. Provisional Application No. 60/412,183 filed Sep. 20, 2002, entitled “Surgical Instrument and Method for Extraction of an Implant”, which is incorporated herein by reference.

### FIELD OF THE INVENTION

The present invention relates generally to the field of surgical instrumentation and methods, and more particularly to instruments and methods for surgical extraction.

### BACKGROUND

In the treatment of diseases, injuries or malformations affecting spinal motion segments, and especially those affecting the intervertebral disc, it has long been known to remove some or all of a degenerated, ruptured or otherwise failing vertebral tissue. In cases involving intervertebral disc tissue that has been removed or is otherwise absent from a spinal motion segment, corrective measures are typically used to ensure proper spacing between the adjacent vertebrae formerly separated by the removed disc tissue.

Various types and configurations of implants have been developed for maintaining proper spacing of the intervertebral disc space. For example, artificial disc devices have been developed for maintaining proper spacing of the intervertebral disc space while allowing a certain degree of relative movement between the adjacent vertebrae. Such devices usually include superior and inferior implant components that are engaged to respective upper and lower vertebrae with certain type of articular element disposed therebetween to allow the adjacent vertebrae to pivot, rotate and/or translate relative to one another.

In some instances, it may become necessary to remove or extract the spinal implant from the intervertebral disc space. For example, the spinal implant may require maintenance or possible replacement by a different type or configuration of implant. Thus, there is a general need in the industry to provide surgical instruments and methods for the extraction of a spinal implant from the intervertebral disc space. The present invention satisfies this need and provides other benefits and advantages in a novel and unobvious manner.

### SUMMARY

The present invention relates generally to instruments and methods for surgical extraction. While the actual nature of the invention covered herein can only be determined with reference to the claims appended hereto, certain forms of the invention that are characteristic of the several embodiments disclosed herein are described briefly as follows.

In one embodiment, a surgical instrument for extracting a prosthetic device includes a distal portion transitionable from an insertion configuration to an extraction configuration, wherein the insertion configuration is adapted for displacement along a portion of a prosthetic device, and the extraction configuration is adapted for engaging and extracting the prosthetic device, and a proximal portion connected to the distal portion.

In another embodiment, an instrument for surgical extraction includes at least one extraction prong wherein the at least one extraction prong comprises a transverse flange, and a mounting portion wherein the at least one extraction prong is secured to the mounting portion.

In a third embodiment, a method for surgical extraction includes inserting a surgical instrument having a distal portion transitionable from an insertion configuration to an extraction configuration; transitioning the distal portion to the extraction configuration; engaging the distal portion with an implant; and exerting an extraction force to extract the implant.

### BRIEF DESCRIPTION OF THE DRAWINGS

FIG. 1 is a side perspective view of a surgical instrument according to one embodiment of the present invention.

FIG. 2 is a perspective view of the distal end portion of the surgical instrument illustrated in FIG. 1.

FIG. 3a is a side cross-sectional view of the distal portion of the surgical instrument illustrated in FIG. 2 in an insertion configuration.

FIG. 3b is a side cross-sectional view of the distal portion of the surgical instrument illustrated in FIG. 2 in an extraction configuration.

FIG. 4 is a view of a mounting block according to one embodiment of the present invention.

FIG. 5 is an end view of the mounting block illustrated in FIG. 4.

FIG. 6 is a cross-sectional view of the mounting block illustrated in FIG. 4, as viewed along line 6-6 of FIG. 4.

FIG. 7 is a view of a first engaging member according to one embodiment of the present invention.

FIG. 8 is a side view of the first engaging member illustrated in FIG. 7.

FIG. 9 is a view of a second engaging member according to one embodiment of the present invention.

FIG. 10 is a side view of the second engaging member illustrated in FIG. 9.

FIG. 11 is a side perspective view of one embodiment of an implant suitable for extraction by the surgical instrument illustrated in FIG. 1.

FIG. 12 is a side perspective view of the distal end portion of the surgical instrument illustrated in FIG. 1 and the implant shown in FIG. 11.

FIG. 13 is a partial sectional view of the implant shown in FIG. 1 disposed between upper and lower vertebrae, with the distal end portions of the first and second engaging members positioned between first and second components of the implant in a compressed, insertion configuration.

FIG. 14 is a partial sectional view of the implant shown in FIG. 11 disposed between the upper and lower vertebrae, with the distal end portions of the first and second engaging members positioned adjacent posterior end surfaces of the implant in an expanded, extraction configuration.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is hereby intended, such alterations and further modifications in the illustrated devices, and such further applications of the principles of the invention as illustrated herein being contemplated as would normally occur to one skilled in the art to which the invention relates.

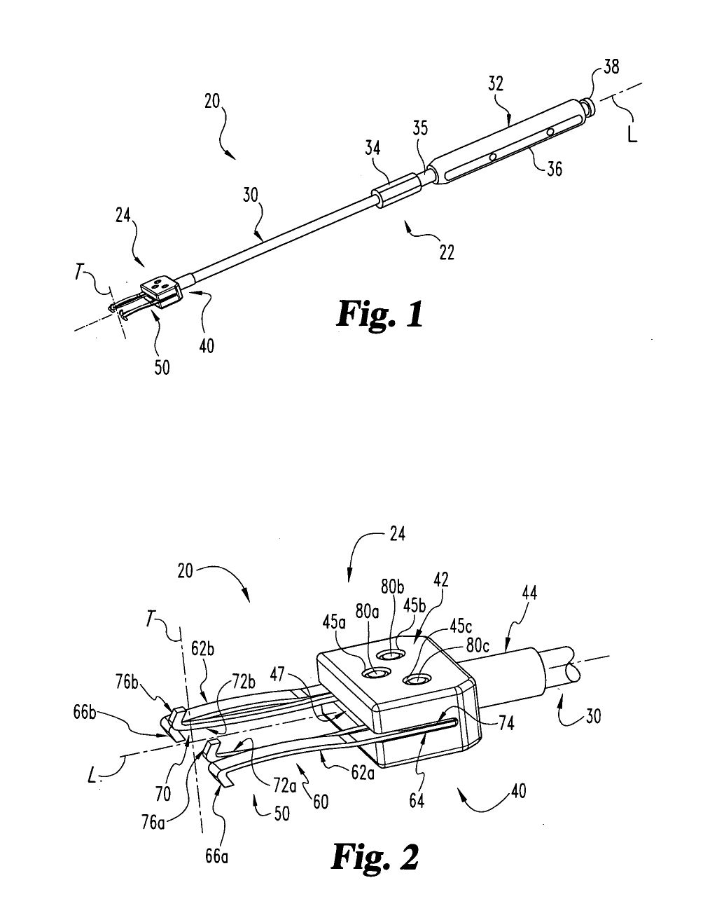

Referring now to FIG. 1, shown therein is a surgical instrument 20 according to one embodiment of the present invention for extraction of an implant. The surgical instrument 20 extends generally along a longitudinal axis L, and comprises a proximal portion, which may be an elongated portion 22, and a distal portion 24. The distal portion 24 is attached to the distal end of the elongated portion 22, and is configured to engage an implant for subsequent extraction, the details of which will be described below. The surgical instrument 20 maybe useful in extracting a spinal implant from a vertebral space, and more specifically from an intervertebral disc space between adjacent vertebral bodies. It should be understood, however, that the surgical instrument 20 may also be used to extract implants from other portions of the spinal column or in applications outside of the spinal field. For example, it may be used to extract any type of implants, prosthetic devices, tissues, or organs from any anatomical region of an animal body.

In one embodiment of the invention, the elongated portion 20 includes a shaft member 30 and a handle member 32. The shaft member 30 and the handle member 32 may comprise a substantially or partially rigid material, such as titanium, stainless steel or other medical grade materials. The shaft member 30 may comprise a variety of configurations, such as a generally linear, axial, angled or curvilinear configuration. The handle member 32 is removably coupled to the proximal end of the shaft member 30 by a coupling member 34.

In one embodiment, the coupling member 34 is integrally formed with the shaft member 30, and comprises an internally threaded sleeve configured to receive a threaded end portion 35 of the handle member 32 therein to removably attach the handle member 32 to the shaft member 30.

In other embodiments, the shaft member 30 and the handle member 32 may be coupled together by other conventional connecting means, or may alternatively be integrally formed as a single-piece, unitary structure.

In one embodiment, the handle member 32 may comprise a gripping portion 36 and a connector portion 38. The connector portion 38 is adapted for connecting various types of instruments or devices to the surgical instrument 20. In one embodiment, the connector portion 38 is a Hudson-type connector; however, it should be understood that other types and configurations of connectors are also contemplated.

In one embodiment, the distal portion 24 of the surgical instrument 20 comprises a mounting portion 40 and an engaging portion 50. The mounting portion 40 serves to couple the engaging portion 50 with the distal end of the shaft member 30. As will be described in details below, the engaging portion 50 is transitionable from an insertion configuration adapted for displacement along a portion of an implant, to an extraction configuration adapted for engaging and extracting the implant from a vertebral space.

In one embodiment, the engaging portion 50 is transitioned from the insertion configuration to the extraction configuration via expansion or displacement of a distal end portion of the engaging portion 50 generally along the transverse axis T.

Referring now to FIG. 2, shown therein are additional details regarding the distal portion 24 of the surgical instrument 20. In one embodiment, the mounting portion 40 generally comprises a mounting block 42 and a connector stem 44. As will be described in greater details below, the mounting block 42 is adapted to support the engaging portion 50, and includes a number of transverse openings 45a-45c extending therethrough and an axial slot 47 extending from the distal end of the block 42 and intersecting the transverse openings 45a-45c. As will be discussed below, the connector stem 44 is adapted for engaging the shaft member 30 to secure the distal portion 24 of the surgical instrument 20 to the elongated portion 22.

In one embodiment, the engaging portion 50 comprises first and second engaging members 60, 70, each extending generally along the longitudinal axis L. The first engaging member 60 includes a first pair of extraction prongs 62a, 62b extending axially from a mounting plate 64. The second engaging member 70 includes a second pair of extraction prongs 72a, 72b extending axially from a mounting plate 74. It should be understood, however, that each of the first and second engaging members 60, 70 may include any number of extraction prongs, including a single extraction prong or three or more extraction prongs. It is also contemplated that the engaging portion 50 may comprise a fewer or greater number of engaging members.

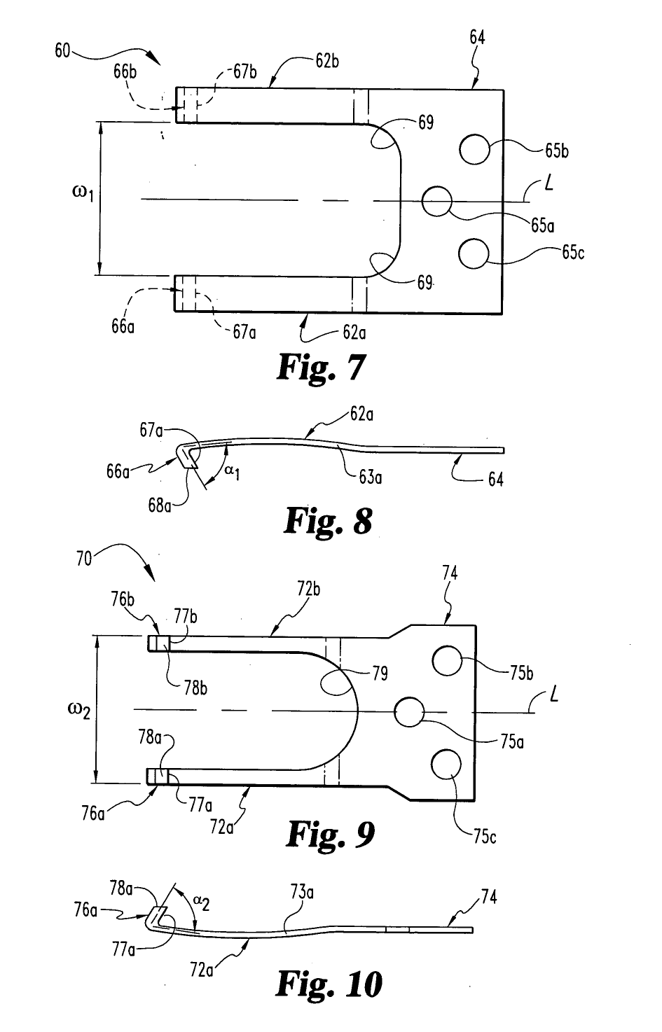

In furtherance of the present example, the mounting plates 64, 74 of the respective engaging members 60, 70 are inserted within the axial slot 47 in the mounting block 45 in an overlapping relationship, with the second pair of extraction prongs 72a, 72b positioned intermediate the first pair of extraction prongs 62a, 62b. In one embodiment, the engaging members 60, 70 are secured to the mounting block 42 via a number of pins or fasteners 80a-80c passing through corresponding ones of the transverse openings 45a-45c in the mounting block 42 and corresponding openings 65a-65c, 75a-75c extending through the mounting plates 64, 74, respectively (FIGS. 7 and 9). In another embodiment, the pins 80a-80c may be replaced with various types of conventional fasteners, such as screws, bolts or rivets, to secure the engaging members 60, 70 to the mounting block 42. In yet another embodiment, the engaging members 60, 70 may be directly attached to the mounting block 42 by any conventional means, such as by welding or by an adhesive. In still another embodiment, the engaging members 60, 70 may be integrally formed with the mounting block 42 to define a single-piece, unitary structure.

In one embodiment, the distal end portions of the extraction prong 62a, 62b may be turned or bent over to define a pair of transverse flanges or lips 66a, 66b. Similarly, the distal end portions of the extraction prong 72a, 72b may be turned or bent over to define a pair of transverse flanges or lips 76a, 76b. As will be discussed below, the transverse flanges 66a, 66b and 76a, 76b may each have a hook-shaped configuration or other shapes adapted to engaging a portion of an implant for subsequent extraction. In one embodiment, the first pair of transverse flanges 66a, 66b and the second pair of transverse flanges 76a, 76b extend in a generally opposite directions, the purpose of which will be discussed below.

The engaging members 60, 70 are at least partially formed of a relatively flexible, resilient material that is capable of being transitioned from a compressed, insertion configuration to an expanded, extraction configuration. In one embodiment, the engaging members 60, 70 comprise type 420 stainless steel. However, it should be understood that other materials are also contemplated, including but not limited to other types of stainless steel, titanium, elastomer, polymer, composite materials or shape memory alloys.

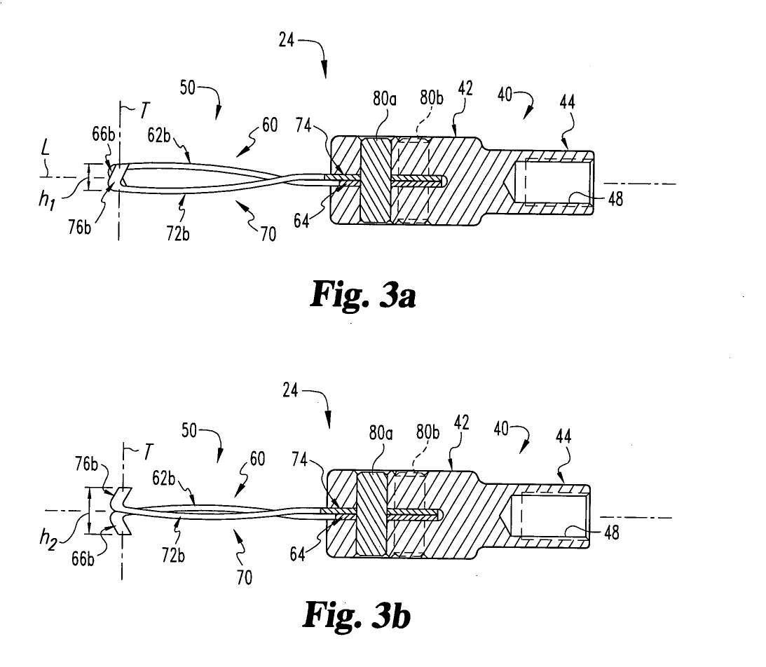

Referring now to FIGS. 3a and 3b, shown therein is the distal portion 24 of the surgical instrument 20, as illustrated in a compressed, insertion configuration and an expanded, extraction configuration, respectively.

Referring specifically to FIG. 3a, the extraction prongs 62a, 62b of the engaging member 60 and the extraction prongs 72a, 72b of the engaging member 70 may be inwardly compressed (toward longitudinal axis L) in the direction of transverse axis T to define the compressed, insertion configuration. In that compressed configuration, the engaging members 60, 70 define a reduced transverse profile having a compressed height h1 to facilitate the insertion of the extraction instrument 20.

Referring specifically to FIG. 3b, when the compression force exerted on the extraction prongs 62a, 62b and 72a, 72b is released, the engaging members 60, 70 are outwardly displaced in the direction of transverse axis T to define the expanded, extraction configuration. In that expanded configuration, the engaging members 60, 70 define an increased transverse profile having an expanded height h2. The increased transverse profile facilitates engagement of the flange portions 66a, 66b of the engaging member 60 and the flange portions 76a, 76b of the engaging member 70 with a corresponding portion of the implant, the details of which will be described below.

As discussed above, the engaging members 60, 70 may comprise a shape-memory material, such as a shape-memory alloy (“SMA”), to aid in transitioning the engaging members 60, 70 from the insertion configuration (FIG. 3a) into the extraction configuration (FIG. 3b). More specifically, SMAs are known to exhibit a characteristic or behavior in which a particular component formed of an SMA is capable of being deformed from an initial “memorized” shape or configuration to a different shape or configuration, and then transitioned back toward the initial, memorized shape or configuration. If the engaging members 60, 70 comprise an SMA material and are compressed to the insertion configuration while at a temperature above the transformation temperatures of the SMA material, the engaging members 60, 70 will automatically recover or transition back toward the extraction configuration when the compression force is removed. This phenomenon is sometimes referred to a stress-induced martensitic (“SIM”) transformation. It will be understood that shape memory alloys and their properties are known in the art, and will only be briefly described herein.

While there are many alloys that exhibit shape-memory or SIM characteristics, one of the more common SMAs is an alloy formed of nickel and titanium. One such well-known SMA is Nitinol, which has proven to be highly effective for instruments and devices used in association with an animal body. Depending on its composition and treatment, transformation temperature range generally may fall between room temperature and normal human body temperature (i.e., about 35-40 degrees Celsius). Moreover, Nitinol has a very low corrosion rate and excellent wear resistance, thereby providing an additional advantage when used in association with the animal body. It should be understood, however, that SMA materials other than Nitinol are also contemplated for use in association with the present invention.

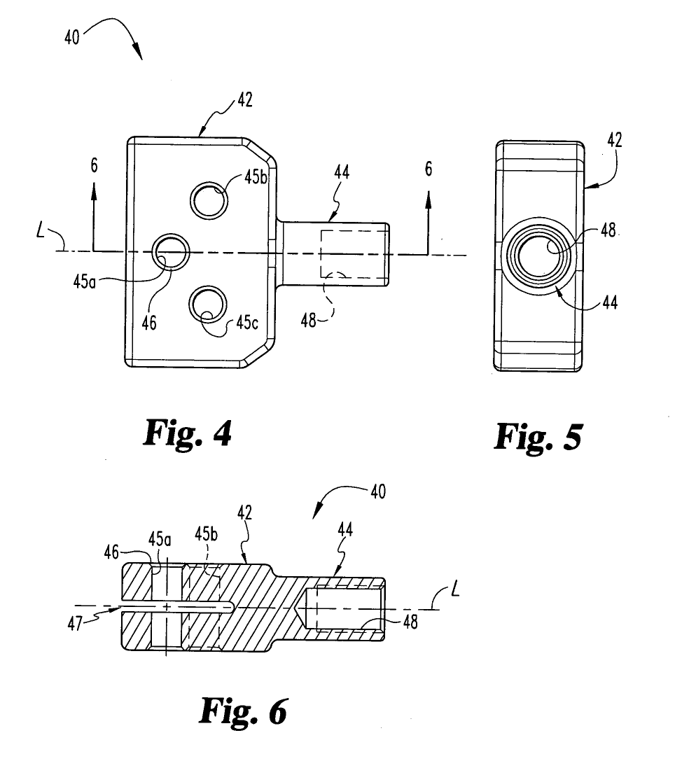

Referring now to FIGS. 4-6, shown therein are additional details regarding the mounting portion 40 of the surgical instrument 20. The mounting portion 40 may comprise a substantially rigid material, such as titanium, stainless steel or other substantially rigid medical grade materials. As discussed above, the mounting portion 40 generally comprises a mounting block 42 and a connector stem 44.

In one embodiment, the mounting block 42 has a generally rectangular configuration; however, other shapes and configuration are also contemplated. The mounting block 42 includes three transverse opening 45a-45c extending therethrough which are sized to receive corresponding ones of the pins 80a-80c therein. In one embodiment, the openings 45a-45c are arranged in a triangular hole pattern. However, it should be understood that other hole patterns are also contemplated. It should also be understood that the mounting block 42 may define any number of transverse openings, including a single opening, two openings or four or more openings.

In furtherance of the embodiment, each of the transverse openings 45a-45c may have an inner diameter substantially equal to the outer diameter of each of the pins 80a-80c. The pins 80a-80c are press fit into the openings 45a-45c to permanently engage the pins 80a-80c within the openings 45a-45c, and to securely attach the engaging members 60, 70 to the mounting block 42. Each end of the openings 45a-45c defines a chamber 46 opening onto the outer surface of the mounting block 42 to facilitate insertion of the pins 80a-80c and/or to aid in the press fitting process. The mounting block 42 may also include an axial slot 47 extending partially therethrough and intersecting each of the transverse openings 45a-45c. The axial slot 47 may have a width sized to snuggly receive the mounting plates 64, 74 of the engaging members 60, 70 therein in an overlapping relationship (FIG. 6).

In one embodiment, the connector stem 44 extends perpendicularly from the mounting block 42 and has a generally cylindrical configuration; however, other shapes and configurations are also contemplated. In the illustrated embodiment, the connector stem 44 and the mounting block 42 are integrally formed to define a single-piece, unitary mounting portion 40. However, it should be understood that the connector stem 44 and the mounting block 42 may be formed separately and attached together by various conventional methods, such as welding or fastening. In the illustrated embodiment, the connector stem 44 is removably coupled to the distal end of the shaft member 30 via a threaded connection. Specifically, the connector stem 44 defines a threaded passage 48 sized to receive a threaded end portion (not shown) of the shaft member 30 therein to removably couple the distal portion 24 of the surgical instrument 20 with the elongated portion 22 (FIG. 1). However, in other embodiments of the invention, the connector stem 44 and the shaft member 30 may be coupled together by other connecting means, or may alternatively be integrally formed as a single-piece, unitary structure.

Referring now to FIGS. 7 and 8, shown therein are additional details regarding the first engaging member 60 of the surgical instrument 20. As discussed above, the first engaging member 60 includes a pair of extraction prongs 62a, 62b extending axially from the mounting plate 64. The mounting plate 64 includes three openings 65a-65c extending therethrough that are arranged in a hole pattern corresponding to the hole pattern of the transverse openings 45a-45c extending through the mounting block 42. In one embodiment, the openings 65a-65c have an inner diameter substantially equal to the outer diameter of the pins 80a-80c. A close match between the openings 65a-65c and the pins 80a-80c (FIGS. 3a and 3b) provides relatively secure and rigid engagement between the first engaging member 60 and the mounting block 42.

In one embodiment, each of the extraction prongs 62a, 62b may have a generally rectangular shape and be arranged in a substantially parallel relationship relative to the other. The extraction prongs 62a, 62b are offset from one another to define an open area therebetween having an inner width w1. In another embodiment, the distal end portions of the extraction prongs 62a, 62b are turned or bent over to define a respective pair of transverse flanges 66a, 66b each having a hook-shaped configuration. Each of the flanges 66a, 66b are arranged at an angle relative to the mounting plate 64. In one embodiment, the angle falls within a range of about 30 degrees to about 90 degrees. In a specific embodiment, the angle α1 may be about 60 degrees. However, it should be understood that other angles of α1 are also contemplated, including angles less than 30 degrees or greater than 90 degrees. The engagement flanges 66a, 66b define inner bearing surfaces or edges 67a, 67b, respectively, each facing toward the mounting plate 64. The engagement flanges 66a, 66b also define end surfaces 68a and 68b, respectively, each of which may be generally parallel to the mounting plate 64. As will be described below, the flanges 66a, 66b, and more specifically the bearing surfaces or edges 67a, 67b, are adapted to engage a corresponding portion of an implant for subsequent extraction of the implant.

As discussed above, the engaging member 60 may comprise at least partially a relatively flexible, resilient material so as to facilitate transformation of the engaging member 60 from the compressed configuration illustrated in FIG. 3a to the expanded configuration illustrated in FIG. 3b. In one embodiment, the extraction prongs 62a, 62b are outwardly biased toward the expanded configuration illustrated in FIG. 3b. In order to further facilitate the transition from the compressed configuration to the expanded configuration, the extraction prongs 62a, 62b may include curved intermediate portions 63a, 63b having a bow-like or arcuate configuration. The intermediate portions 63a, 63b may function similar to that of a leaf spring, storing energy upon the imposition of a compression force onto the extraction prongs 62a, 62b and discharging the energy upon the release of the compression force to expand the extraction prongs 62a, 62b. In one embodiment, the interface between each of the extraction prongs 62a, 62b and the mounting plate 64 defines a rounded corner 69. The rounded corners 69 serve to strengthen the interconnection between the extraction prongs 62a, 62b and the mounting plate 64, and minimize stress concentrations during compression and expansion of the extraction prongs 62a, 62b and/or to further facilitate transitioning of the extraction prongs 62a, 62b from the compressed configuration to the expanded configuration.

Referring to FIGS. 9 and 10, shown therein are additional details regarding the second engaging member 70 of the surgical instrument 20 according to one embodiment of the present invention. As discussed above, the second engaging member 70 may include a pair of extraction prongs 72a, 72b extending axially from the mounting plate 74. The mounting plate 74 may include three openings 75a-75c extending therethrough, which are arranged in a hole pattern corresponding to the hole pattern of the transverse openings 45a-45c extending through the mounting block 42. In one embodiment, each of the openings 75a-75c may have an inner diameter that is substantially equal to the outer diameter of each of the pins 80a-80c. A close tolerance between the openings 75a-75c and the pins 80a-80c (FIGS. 3a and 3b) provides relatively secure and rigid engagement between the second engaging member 70 and the mounting block 42.

In one embodiment, the extraction prongs 72a, 72b have generally rectangular shapes and are arranged in a substantially parallel relationship relative to one another. The extraction prongs 72a, 72b are offset from one another to define an open area therebetween. The extraction prongs 72a, 72b of the engaging member 70 define an outer width w2 that is sized somewhat less than the inner width w1 between the extraction prongs 62a, 62b of the engaging member 60. In this manner, as illustrated in FIG. 2, the extraction prongs 72a, 72b may be positioned within the open area between the extraction prongs 62a, 62b to nest the inner extraction prongs 72a, 72b between the outer extraction prongs 62a, 62b.

In another embodiment, the distal end portions of the extraction prong 72a, 72b are turned or bent over to define a respective pair of transverse flanges 76a, 76b, each having a hook-shaped configuration. The transverse flanges 76a, 76b are arranged at an angle α2 relative to the mounting plate 74. In one embodiment, the angle α2 falls within a range of about 30 degrees to about 90 degrees. In a specific embodiment, the angle α2 may be about 60 degrees. However, it should be understood that other angles α2 are also contemplated, including angles less than 30 degrees or greater than 90 degrees. The flanges 76a, 76b define inner bearing surfaces or edges 77a, 77b, respectively, that face toward the mounting plate 74. The engagement flanges 76a, 76b also define end surfaces 78a, 78b that may be arranged generally parallel with the mounting plate 74. As will be described below, the flanges 76a, 76b, and more specifically the bearing surfaces or edges 77a, 77b, may be adapted to engage a corresponding portion of an implant for subsequent extraction of the implant from an intervertebral disc space.

As discussed above, the engaging member 70 may comprise at least partially a relatively flexible, resilient material to facilitate transformation of the engaging member 70 from the compressed configuration illustrated in FIG. 3a to the expanded configuration illustrated in FIG. 3b. In one embodiment, the extraction prongs 72a, 72b are outwardly biased toward the expanded configuration illustrated in FIG. 3b. In order to further facilitate transformation from the compressed configuration to the expanded configuration, the extraction prongs 72a, 72b may include curved intermediate portions 73a, 73b, each having a bow-like or arcuate configuration. Like the intermediate portions 63a, 63b of the extraction prongs 62a, 62b, the intermediate portions 73a, 73b may also function similar to that of a leaf spring, storing and releasing energy to facilitate transitioning of the extraction prongs 72a, 72b from the insertion configuration to the extraction configuration illustrated in FIG. 3b. In one embodiment, the interface between the extraction prongs 72a, 72b and the mounting plate 74 defines a concave recess 79. The concave recess 79 serves to strengthen the interconnection between the extraction prongs 72a, 72b and the mounting plate 74, to minimize stress concentrations during compression and expansion of the extraction prongs 72a, 72b and/or to further facilitate transitioning of the extraction prongs 72a, 72b from the compressed configuration to the expanded configuration.

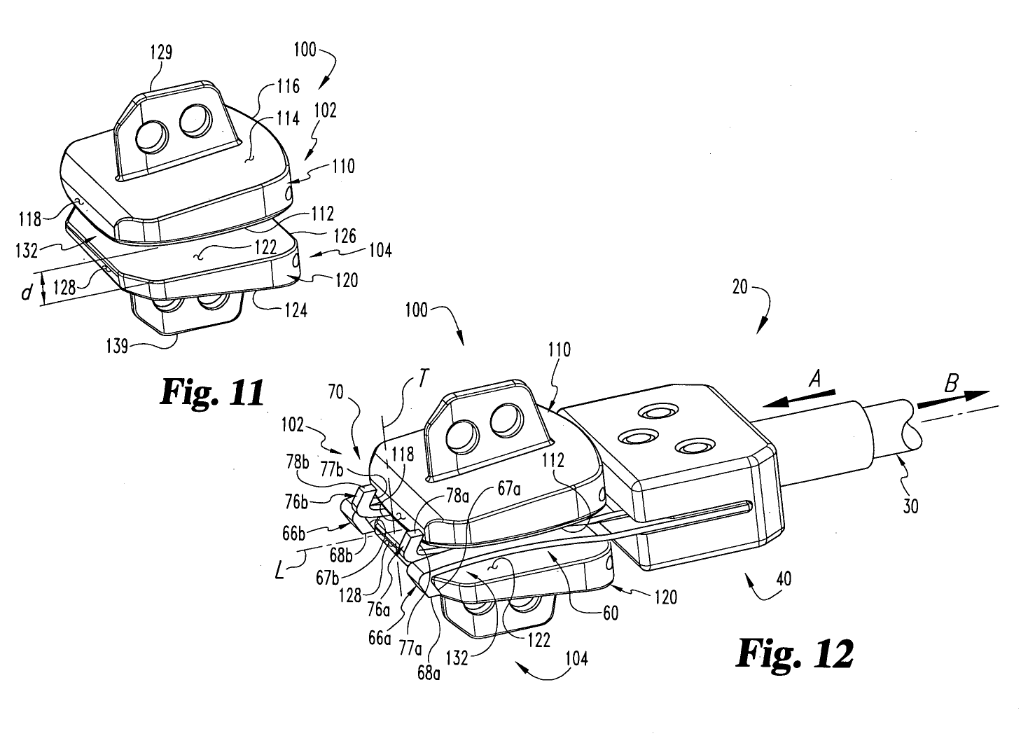

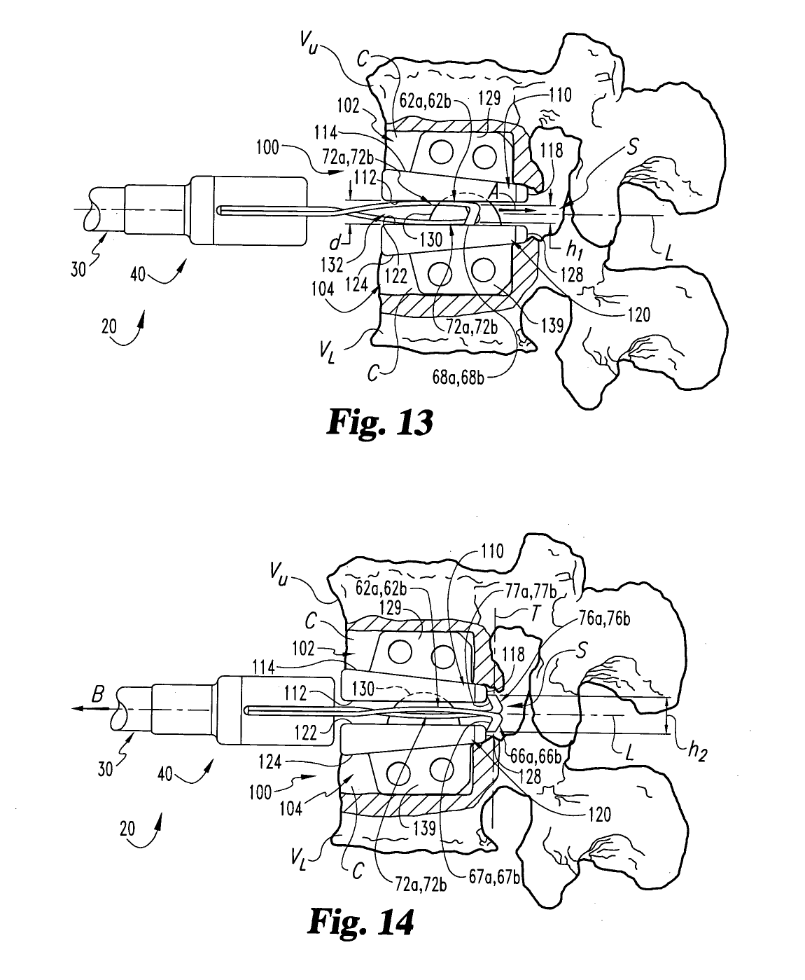

Referring to FIG. 11, shown therein is one embodiment of a spinal implant 100 suitable for extraction from a vertebral space by the surgical instrument 20. The implant 100 is configured for implantation within an intervertebral disc space S between upper and lower vertebrae VU, VL (FIGS. 13 and 14) and includes a superior component 102 and an inferior component 104. In one embodiment of the invention, the superior and inferior components 102, 104 comprise separate or discrete components of the implant 100. However, it should be understood that the superior and inferior components 102, 104 may alternatively be integrally formed to define a single-piece, unitary implant 100. In one embodiment, the superior and inferior components 102, 104 cooperate to form an articulating prosthetic joint. In a specific embodiment, the articulating joint is capable of providing relative pivotal and rotational movement between the adjacent vertebral bodies to maintain or restore motion substantially similar to the normal bio-mechanical motion provided by a natural intervertebral disc. However, it should be understood that other types of articulating or non-articulating implants are also contemplated for use in association with the present invention.

In one embodiment of the invention, the superior implant component 102 includes a support plate 110 having an inner surface 112, an outer surface 114, and anterior and posterior end surfaces 116, 118 extending between the inner and outer surfaces 112, 114. Similarly, the inferior implant component 104 includes a support plate 120 having an inner surface 122, an outer surface 124 and anterior and posterior end surfaces 126, 128 extending between the inner and outer surfaces 122, 124. A spherical-shaped ball or projection 130 extends from the inner surface 122 of the inferior component 104 (FIG. 13), which is at least partially engaged within a spherical-shaped recess (not shown) extending from the inner surface 112 of the superior component 102. The spherical-shaped projection 130 and the spherical-shaped recess (not shown) cooperate to allow the superior and inferior components 102, 104 to articulate relative to one another. The inner surfaces 112, 122 of the superior and inferior implant components 102, 104 are separated by a distance d so as to define a gap or passage 132 therebetween. As will be described below, the gap 132 is sized to allow for insertion of the engaging portion 50 of the surgical instrument 20 therein when the surgical instrument 20 is in the insertion configuration (FIGS. 3a and 13).

In furtherance of the example, the outer surfaces 114, 124 of the superior and inferior support plates 110, 120 are adapted to bear against the vertebral endplates of the upper and lower vertebrae VU, VL. In one embodiment, the outer surfaces 114, 124 are sized and shaped to extend substantially entirely across and along the intervertebral disc space S. In another embodiment, the outer surfaces 114, 124 are angled relative to the respective inner surfaces 112, 122 to accommodate for the particular lordotic angle between the upper and lower vertebrae VU, VL. In yet another embodiment, a flange member or keel 129, 139 extends from the respective outer surfaces 114, 124 of the superior and inferior support plates 110, 120. The keels 129, 139 are sized and shaped for disposition within preformed slots or channels C formed through and along the endplates of the upper and lower vertebrae VU, VL (FIGS. 13 and 14) to stabilize the implant within the intervertebral disc space S. Each of the keels 129, 139 defines a number of openings extending therethrough to provide opportunity for bone through-growth to enhance fixation of the spinal implant 100 to the upper and lower vertebrae VU, VL.

Although a specific embodiment of a spinal implant 100 has been illustrated and described herein, it should be understood that other sizes, shapes and configurations of implants are also contemplated. For example, another embodiment of a spinal implant suitable for use in association with the present invention is illustrated and described in U.S. patent application Ser. No. 10/042,589 to Eisermann et al., entitled “Intervertebral Prosthetic Joint” and filed on Jan. 9, 2002, the contents of which are incorporated herein by reference.

Referring to FIG. 12, shown therein is the surgical instrument 20 engaged with the spinal implant 100 according to one embodiment of the present invention. As will be described below, the extraction prongs 62a, 62b and 72a, 72b of the respective engaging members 60, 70 are initially inwardly compressed toward one another to define the insertion configuration illustrated in FIG. 3a. While in this reduced profile insertion configuration, the engaging members 60, 70 are displaced through the gap 132 between inner surfaces 112, 122 of the implant support plates 110, 120 generally along the longitudinal axis L in the direction of arrow A. Once the distal end portions of the engaging members 60, 70 pass beyond the posterior surfaces 118, 128 of the inferior and superior implant components 102, 104, the engaging members 60, 70 will automatically transition to the expanded, extraction configuration illustrated in FIGS. 3b and 12. During the transitioning, the transverse flanges 66a, 66b and 76a, 76 are outwardly displaced in generally opposite directions along the transverse axis T. As a result, the inner bearing surfaces 67a, 67b of the engaging member 60 are positioned adjacent the posterior end surface 128 of the inferior implant component 104, and the inner bearing surfaces 77a, 77b of the engaging member 70 are positioned adjacent the posterior end surface 118 of the superior implant component 102. The surgical instrument 20 is then displaced generally along the longitudinal axis L in the direction of arrow B to engage the bearing surfaces 77a, 77b and 67a, 67b securely against the posterior end surfaces 118, 128 of the inferior and superior implant components 102, 104.

Referring to FIGS. 13 and 14, shown therein is the exemplary spinal implant 100 inserted within an intervertebral disc space S between the upper and lower vertebrae VU, VL, with the outer surfaces 114, 124 of the inferior and superior support plates 110, 120 engaged against the vertebral endplates and with the keels 129, 139 positioned within the channels C formed through and along the vertebral endplates.

In this example, the spinal implant 100 is positioned within the intervertebral disc space S with the superior and inferior implant components 102, 104 disposed in a vertical or stacked arrangement extending between the upper and lower vertebrae VU, VL. However, it should be understood other arrangements are also contemplated. For example, in another embodiment, the spinal implant may comprise a pair of bi-lateral implant components disposed in a horizontal or side-by-side arrangement within the intervertebral disc space S. In one such alternative embodiment, the spinal implant may comprise a pair of fusion cages or spacers positioned bi-laterally within the intervertebral disc space S and separated by a distance to define a gap or passage therebetween sized to receive the engaging members 60, 70 of the surgical instrument 20 therethrough when in the compressed, insertion configuration. It should be understood that other types, configurations and arrangements of implants are also contemplated for use in association with the present invention.

FIG. 13 illustrates the surgical instrument 20 as it is being axially displaced in a posterior direction along the gap 132 between the inferior and superior components 102, 104 of the implant 100 according to one embodiment of the present invention. FIG. 14 illustrates engagement of the surgical instrument 20 with the inferior and superior components 102, 104 for extraction of the implant 100 from the intervertebral disc space S in an anterior direction. In the illustrated embodiment of the invention, the surgical instrument 20 is used to extract the spinal implant 100 from the intervertebral disc space S via an anterior approach. However, it should be understood that the surgical instrument 20 may alternatively be used to extract the spinal implant 100 from the intervertebral disc space S via a posterior approach, a lateral approach, or other surgical approaches known to those skilled in the art.

Referring to FIG. 13, in one embodiment, prior to inserting the engaging members 60, 70 within the gap 132 between the inferior and superior implant components 102, 104, the extraction prongs 62a, 62b and 72a, 72b may be inwardly compressed toward one another to the insertion configuration. When in the compressed configuration, the engaging members 60, 70 define a reduced profile having a compressed height h1 substantially equal to the distance d between the inner support plate surfaces 112, 122. While in this reduced profile insertion configuration, the extraction prongs 62a, 62b and 72a, 72b may be displaced through the gap 132 in the direction of arrow A generally along the longitudinal axis L.

In furtherance of the example, during displacement along the gap 132, the engaging members 60, 70 may be maintained in the compressed state via engagement of distal end surfaces 68a, 68b of the flanges 66a, 66b against the inner support plate surface 112, and via engagement of distal end surfaces 78a, 78b of flanges 76a, 76b against the inner support plate surface 122. Additionally, as the engaging members 60, 70 are displaced along the gap 132, the spherical-shaped projection 130 extending from the inner support plate surface 122 may pass through the open area between the extraction prongs 72a, 72b of the engaging member 70, thereby allowing the distal end portions of the engaging members 60, 70 to pass entirely through the gap 132.

Referring to FIG. 14, in one embodiment, once the transverse flanges 66a, 66b and 76a, 76b of the respective engaging members 60, 70 are positioned beyond the posterior edges of the inner support plate surfaces 112, 122, the engaging members 60, 70 may automatically transition to the expanded, extraction configuration. More specifically, when the flanges 66a, 66b and 76a, 76b are positioned beyond the support plates 110, 120, the distal end surfaces 68a, 68b of the transverse flanges 66a, 66b and the distal end surfaces 78a, 78b of transverse flanges 76a, 76b will disengage the inner support plate surfaces 112, 122. Since the engaging members 60, 70 are biased toward the extraction configuration, the prongs 62a, 62b and 72a, 72b will automatically expand in an outward direction along the transverse axis T. When in the expanded configuration, the engaging members 60, 70 define an increased profile having an expanded height h2 that is greater than the distance d between the inner support plate surfaces 112, 122. As a result, the inner bearing surfaces 67a, 67b of the engaging member 60 will be positioned adjacent the posterior end surface 128 of the inferior implant component 104, and the inner bearing surfaces 77a, 77b of the engaging member 70 will be positioned adjacent the posterior end surface 118 of the superior implant component 102.

In furtherance of the example, once the engaging members 60, 70 are transitioned into the expanded configuration, an extraction force may be exerted onto the surgical instrument 20 in the direction of arrow B, which may be transmitted through the shaft member 30 to the engaging member 60, 70, to extract the implant from the intervertebral disc space S. Notably, since the surgical instrument 20 engages both the superior and inferior implant components 102, 104, the implant 100 may be extracted from the intervertebral disc space S as a single unit. Extraction of the entire implant 100 eliminates the requirement of having to distract the intervertebral disc space S to individually remove the inferior and superior implant components 102, 104. Extraction of the implant 100 as a single unit also avoids stretching of the ligaments that extend between the upper and lower vertebrae VU, VL. However, it is understood that the inferior and superior implant components 102 and 104 may be extracted separately.

Referring back to FIG. 1, in one embodiment, the extraction force exerted onto the surgical instrument 20 may be generated by an impact or slap hammer (not shown) or another type of impact device. The slap hammer may be attached to the handle member 32 via the Hudson-type connector portion 38. Alternatively, the handle member 32 may be removed from the instrument 20, and the slap hammer may be connected to the shaft member 30 via the internally threaded coupling member 34. Slap hammers are well known in the art and typically including a weight that freely slides along the length of a guide rod with a stop member secured to the end of the guide rod. Impacting the weight against the stop member in turn exerts a controlled force onto the shaft member 30, which in turn is transmitted to the engaging members 60, 70 to exert an extraction force onto the spinal implant 100. It should be understood, however, that other devices and techniques may be used to exert a force onto an implant to facilitate its removal. For example, in an alternative embodiment, a surgeon may manually grasp the handle member 32 and exert a pulling force in the direction of the axis L to extract the implant.

Although only a few exemplary embodiments of this invention have been described above in details, those skilled in the art will readily appreciate that many modifications are possible in the exemplary embodiments without materially departing from the novel teachings and advantages of this invention. Also, features illustrated and discussed above with respect to some embodiments can be combined with features illustrated and discussed above with respect to other embodiments. Accordingly, all such modifications are intended to be included within the scope of this invention.

What is claimed is:

1. A surgical instrument for extracting a prosthetic device, comprising:
a distal portion transitionable from an insertion configuration to an extraction configuration, wherein the insertion configuration is adapted for displacement along a portion of a prosthetic device, and the extraction configuration is adapted for engaging and extracting the prosthetic device; and
a proximal portion connected to the distal portion.

2. The surgical instrument of claim 1 wherein the distal portion comprises at least one engaging member.

3. The surgical instrument of claim 2 wherein the at least one engaging member comprises a flexible material that is capable of being transferred from the insertion configuration to the extraction configuration.

4. The surgical instrument of claim 2 wherein the at least one engaging member comprises stainless steel.

5. The surgical instrument of claim 2 wherein the at least one engaging member is secured to a mounting block.

6. The surgical instrument of claim 2 wherein each of the at least one engaging member comprises at least one extraction prong.

7. The surgical instrument of claim 6 wherein each of the at least one extraction prong comprises a transverse flange.

8. The surgical instrument of claim 7 wherein the transverse flange comprises a hook-shaped configuration.

9. The instrument of claim 7 wherein at least two of the transverse flanges extend in generally opposite directions.

10. The instrument of claim 7 wherein at least two of the transverse flanges extend in generally parallel directions.

11. An instrument for surgical extraction, comprising:
at least one extraction prong wherein the at least one extraction prong comprises a transverse flange; and
a mounting portion wherein the at least one extraction prong is secured to the mounting portion.

12. The instrument of claim 11 wherein the at least one extraction prong is transitionable from an insertion configuration to an extraction configuration.

13. The instrument of claim 11 wherein the transverse flange defines a reduced transverse profile for an insertion configuration.

14. The surgical instrument of claim 11 wherein the transverse flange comprises a hook-shaped configuration.

15. The surgical instrument of claim 11 wherein the at least one extraction prong comprises a flexible material that is capable of being transferred from an insertion configuration to an extraction configuration.

16. The surgical instrument of claim 11 wherein the at least one extraction prong comprises stainless steel.

17. The surgical instrument of claim 11 wherein the mounting portion comprises a mounting block.

18. A method for surgical extraction, comprising:
inserting a surgical instrument having a distal portion transitionable from an insertion configuration to an extraction configuration;
transitioning the distal portion to the extraction configuration;
engaging the distal portion with an implant; and
exerting an extraction force to extract the implant.

19. The method of 18 further comprising displacing the distal portion along at least a portion of the implant.

20. The method of 18 wherein the distal portion comprises at least one transverse flange.
